(19) United States
(12) Patent Application Publication
(10) Pub. No.: US 2004/0193103 A1
(43) Pub. Date: Sep. 30, 2004

(54) Apparatus and method for sclerosing the wall of a blood vessel

(75) Inventor: Ravi Kumar, Briarcliff Manor, NY (US)

Correspondence Address: William L. Botjer, PO Box 478, Center Moriches, NY, 11934, US

(73) Assignee: ARVIK ENTERPRISES, LLC

(21) Appl. No.: 10/678,329

(22) Filed: Oct. 3, 2003

Related U.S. Application Data
(63) Continuation-in-part of application No. 10/400,901, filed on Mar. 27, 2003.

(51) Int. Cl. A61M 3/00

(52) U.S. Cl. 604/43; 602/53

(57) ABSTRACT

A catheter system is provided for use in sclerotherapy of varicose veins. The catheter system comprises a multi-lumen tube, a tip device and a fluid porting device. The multi-lumen tube comprises a coaxial center lumen and three equally spaced outer lumens in the annulus between the central lumen and the outer wall of the multi-lumen tube. The multi-lumen tube is used for carrying sclerosing agent and irrigation fluid and for providing suction at the affected site in the vein. The catheter device is introduced inside the vein and the tip device is positioned at the affected site. Pressure is applied over the affected vein using a pressure applying device. The catheter device is slowly withdrawn while continuously releasing the sclerosing agent from the tip device. This helps in achieving uniform sclerosis of the affected vein.

This application is a continuation-in-part of U.S. patent application Ser. No. 10/400,901, titled “Vein Compressor Device”, filed on Mar. 27, 2003, the complete disclosure of which is hereby incorporated by reference herein.

### BACKGROUND

The present invention relates to medical apparatus for treatment and correction of varicose veins. More particularly, the present invention relates to a minimally invasive procedure using a catheter system for the treatment of varicose veins.

The venous system of the lower limbs is divided into three categories: superficial veins, deep veins and communicating (also called perforating) veins. The superficial veins run close to the surface and return blood from skin and superficial structures. The primary superficial veins in the leg are the long saphenous vein (also called great saphenous vein) and the short saphenous vein. The deep veins lie within the deep fascia of the lower limb and these accompany the arteries. The communicating veins connect the superficial veins to the deep veins.

All veins in the lower limbs contain valves every few centimeters. This ensures that the blood flows only in one direction, i.e., either from the superficial veins to the deep veins or towards the heart. The venous valves are usually bicuspid valves and each cusp of the bicuspid valves forms a sack or a reservoir for blood. Under the pressure of blood, this arrangement of bicuspid valves forces the free surfaces of the cusps together to prevent retrograde flow of the blood into the vein.

There are various factors that can cause failure of the bicuspid valve closure, thereby, making them faulty. An incompetent valve is a valve that is unable to close because the cusps do not form a proper seal and hence are unable to prevent the retrograde flow of blood. This causes elevated pressure in the superficial venous system. Initially a single venous valve may fail, thereby, creating high-pressure reflux between the deep and the superficial venous systems. This high-pressure reflux causes local dilatation that leads to sequential failure of other nearby valves in the superficial veins.

Valve failure in superficial veins leads to the formation of dilated and tortuous (i.e., winding, twisted or crooked) superficial veins that are then referred to as varicose veins. Varicose veins usually do not cause fatal complications and patients suffering from varicose veins complain primarily of leg fatigue, dull aching pains, ankle swelling, and ulcerations. And, the highly visible unattractive rope like varicosities and reddish skin blotches may cause considerable distress to both men and women. Hence, all these effects of varicose veins may significantly decrease the quality of life of the patient.

There are many treatment modalities available for the treatment of varicose veins. The treatment of varicose veins aims at correcting the underlying defect, and removing or closing down points of reflux that connect the deep venous system with the superficial venous system. The defective system can either be surgically removed or the affected veins can be ablated (i.e., removal by “vaporizing” or “melting, etc.), to close the venous channel.

The standard surgical treatment for removing the varicose veins involves surgical stripping that uses an internal stripping tool and an “invagination” technique to invert the vessel and pull it through itself. The surgical stripping procedure, for removing varicosities in the long saphenous vein, is an invasive procedure. It may sometimes involve making as many as 40 incisions of varying size to treat all the varicosities. This procedure is very painful and requires significant anesthesia. Surgical stripping also has an associated risk of injuring the nerves that accompany the superficial veins.

Ablation of the affected veins offers a less invasive alternative, and is helpful in the improvement of venous circulation. This ablation may be achieved by impinging laser or radio frequency waves or by destroying venous channels or by injecting a suitable set of sclerosing substances (also called sclerosing agents).

Laser therapy is a thermal ablation technique that uses a laser fiber placed inside the vein to destroy the vascular endothelium. Similarly, Radio Frequency (RF) ablation is a thermal ablation technique that uses a RF catheter placed inside the vein to heat the vessel wall and its surrounding tissues. The tissue heating causes thermal damage to the endothelium that results in closure of the vessel.

Laser and RF ablation procedures have several disadvantages. The RF and laser treatments are quite slow and painful. The patient undergoing treatment must be sufficiently anesthetized in the region that covers the entire length of the veins. In addition, to treat an extensive disease, repositioning of these catheters is time consuming, thereby, requiring anesthesia for a prolonged period. Moreover, great care has to be taken to avoid thermal damage and burns to the surrounding tissue, especially to the nerves accompanying these veins.

Ablation of the affected vein can also be achieved through chemical sclerosis. This procedure is called sclerotherapy (and is called phlebosclerosis). Sclerotherapy is the most widely used medical procedure for ablation of varicose veins. In this procedure, a sclerosing substance is injected into the affected vein to produce endothelial destruction. The damaged endothelium leads to the closure of the affected vein.

For effective sclerotherapy, it is necessary to evenly dispense the sclerosing agent inside the affected vein. Injection of the sclerosing agent into the smaller veins is very effective and easy to perform. However, this procedure is not very effective for sclerosing varicosities in larger veins. In larger veins, the volume of blood is substantially high and this results in quick dilution of the sclerosing agent. Consequently, the sclerosis is only achieved in the vicinity of the injection site. Hence, if this procedure is continued at different sites, the corresponding vein may get disfigured. Moreover, this procedure also carries the risk of using increasing amounts of the sclerosing agent and using more potent solution of sclerosing agents may be toxic. Furthermore, there is an associated risk of leakage of sclerosing agent into the surrounding tissue, thereby, staining this tissue. Finally, the sclerosing agent may also be accidentally injected into an artery that may result in the development of fatal thromboembolism.

It can be readily appreciated that an endovenous catheter system would be advantageous in carrying out sclerosis of major varicose veins. This is because, in this case, the endovenous catheter can be placed directly inside the lumen of the affected major vein for carrying out sclerosis. Keeping this in view, there are quite a few patents that describe the use of endovenous catheters for the treatment of varicose veins, and some of these are briefly discussed below.

U.S. patent application Ser. No. 2003/0120256 titled, “Methods And Apparatus For Sclerosing The Wall Of A Varicose Vein”, describes a catheter-based system for sclerotherapy of varicose veins. The catheter consists of an inflatable tip and a multi-lumen coaxial tube. The multi-lumen tube comprises of an inner, an intermediate, and an outer tube. The method of using the apparatus includes deployment of the inner and the intermediate tube in the vessel to be sclerosed. The outer tube is filled with sclerosing agent. With the movement of a plunger attached to one end of the outer tube, the sclerosing agent is infused in the affected vein.

U.S. patent application Ser. No. 4,795,438 titled, “Methods And Apparatus For Forming A Restriction In A Vessel, Duct Or Lumen”, describes the use of a flexible, power driven catheter for carrying out vessel restriction. The vessel restriction is achieved at the affected tissue site by the working head (of the catheter) achieving sclerosis either by using its mechanical action or by injection of sclerosing agent from the apparatus.

The above-mentioned devices suffer from one or more of the following limitations. Firstly, the utility of the above-mentioned catheters is limited by the catheter design. The design of these catheters is such that it allows limited flexibility. This may hinder their introduction into the tortuous veins. Secondly, the complex design of these catheters makes them difficult to use and may substantially increase the cost of the catheter. Thirdly, these catheters demand great care and expertise while carrying out the sclerosis in the affected vein. Furthermore, these catheters may cause venous thrombosis.

In light of the above drawbacks, there exists a need for a minimally invasive catheter system for effective treatment of varicose veins in the lower limb. The catheter needs to be simple in design and easy to manufacture. Furthermore, there is a need for a catheter system that is convenient to use. Moreover, there is a need for a cost effective catheter system.

### SUMMARY

An object of the invention is to provide a catheter system for minimally invasive treatment of varicose veins, using sclerotherapy.

Another object of the invention is to provide a catheter system for the treatment of the varicose veins wherein the catheter system has increased flexibility and is easy to use.

Yet another object of the invention is to provide methods and apparatus for treatment of the varicose veins wherein the wall of the vein is evenly sclerosed,

Still another object of the invention is to provide a catheter system for treatment of the varicose veins wherein the catheter system is simple in design and easy to manufacture.

In accordance with these objects, a catheter system is provided for use in sclerotherapy of varicose veins. The catheter system comprises a multi-lumen tube, a tip device and a fluid porting device. The multi-lumen tube has a coaxial center lumen and three equally spaced outer lumens in the annulus between the center lumen and the outer wall of the multi-lumen tube. The central lumen is used for carrying the sclerosing agent into the affected vein. The central lumen is also used for introducing the catheter inside the affected vein by passing it over a guide wire. The three outer lumens are used for carrying out irrigation and suction inside the vein. The tip device is attached to the center lumen at one end of the multi-lumen tube, and is used to infuse the sclerosing agent inside the vein. The fluid porting device is attached at the other end of the multi-lumen tube and provides access for introducing and sucking fluids through the lumens.

In order to treat a varicose vein problem in a long saphenous vein using the catheter system, the catheter system is introduced into the long saphenous vein through an incision made in the long saphenous vein at the groin level. The catheter device is allowed to reach the affected site inside the vein. During this process, pressure is applied over the long saphenous vein using a pressure applying device. Thereafter, continuous irrigation and suction is carried out inside the vein. Subsequently, the catheter system is slowly withdrawn from the vein, while maintaining continuous release of the sclerosing agent from the tip device. This helps in achieving uniform sclerosis of the affected vein. Finally the catheter device is fully withdrawn from the vein. The wound is closed and the patient is discharged with the pressure applying device in place over the treated area.

Additional objects and advantages of the invention will become apparent to those skilled in the art upon reference to the detailed description taken in conjunction with the provided figures.

### BRIEF DESCRIPTION OF THE DRAWINGS

The various embodiments of the invention will hereinafter be described in conjunction with the appended drawings provided to illustrate and not to limit the invention, wherein like designations denote like elements, and in which:

FIG. 1 is a sectional view of the catheter system, in accordance with one embodiment of the present invention;

FIG. 2 is a cross-sectional view of the multi-lumen tube, in accordance with one embodiment of the present invention;

FIG. 3 is an isometric view of the tip device coupled to the multi-lumen tube, in accordance with one embodiment of the present invention;

FIG. 4 is a representation of the fluid porting device coupled to the multi-lumen tube, in accordance with one embodiment of the present invention;

FIG. 5 is a flowchart illustrating the method of performing sclerotherapy in a long saphenous vein (LSV) using the catheter system;

FIG. 6 is a flowchart illustrating the method of inserting the catheter system in the LSV;

FIG. 7 is a flowchart illustrating the method of operating the catheter system while sclerosis is being performed; and

FIG. 8 is a flowchart illustrating the method of withdrawing the catheter system from LSV.

### DESCRIPTION OF PREFERRED EMBODIMENTS

The present invention discloses an apparatus and a method for treatment of varicose veins. The treatment of varicose veins is carried out by sclerosing the affected blood vessel. The disclosed system comprises a catheter system that is used for infusing sclerosing agent into the affected blood vessel.

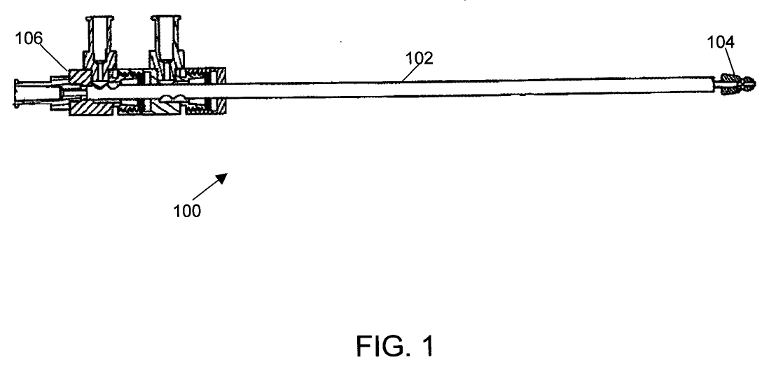

FIG. 1 is a sectional view of the catheter system, in accordance with one embodiment of the present invention. The catheter system 100 is hereinafter referred to as catheter 100. Catheter 100 comprises three distinct parts that include a multi-lumen tube 102, a tip device 104, and a fluid porting device 106. Tip device 104 is attached to multi-lumen tube 102 at the distal end of multi-lumen tube 102. The distal end of multi-lumen tube 102 is the end that is away from the surgeon while performing the surgery, i.e. the end that is first inserted into the affected blood vessel. Fluid porting device 106 is attached to multi-lumen tube 102 at the proximal end of multi-lumen tube 102. The proximal end of multi-lumen tube 102 is the end that is close to the surgeon while performing the surgery.

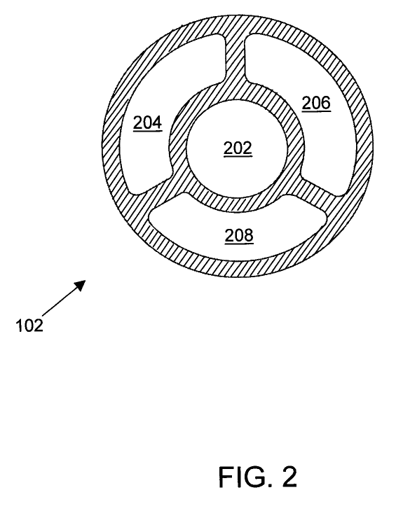

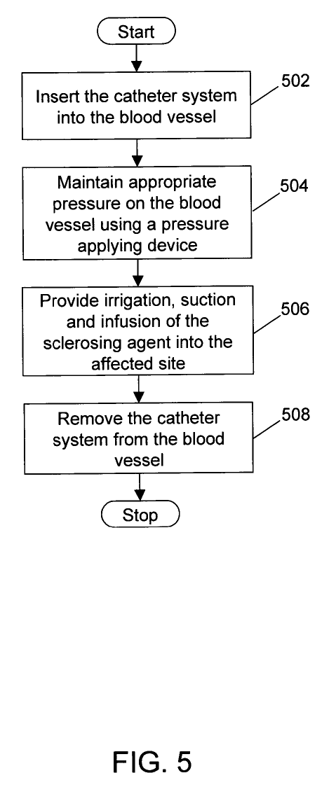

FIG. 2 is cross-sectional view of multi-lumen tube 102, in accordance with one embodiment of the present invention. Multi-lumen tube 102 comprises a center lumen 202 and three outer lumens 204, 206 and 208. Center lumen 202 is coaxial with multi-lumen tube 102. Center lumen 202 is used to carry the sclerosing agent from fluid porting device 106 to tip device 104. The sclerosing agent used in the present invention may be sodium morruate, sodium tettradycol, hypertonic solution and the like. Center lumen 202 is also used for accommodating a guide wire when catheter 100 is inserted in the affected blood vessel. The exact manner in which catheter 100 is used to carry out sclerosis is described later in conjunction with FIG. 5 to FIG. 8.

Outer lumens 204, 206 and 208 are present in the annulus between center lumen 202 and the outer wall of multi-lumen tube 102. Outer lumens 204, 206 and 208 are separated by radial walls in the annulus. Center lumen 202 protrudes out of multi-lumen tube 102 on both proximal and distal ends of multi-lumen tube 100. In one embodiment of the present invention, outer lumens 204, 206 and 208 are equally spaced in the annulus. Outer lumen 204 is used for carrying irrigation fluids from fluid porting device 106 to the distal end of multi-lumen tube 102. Irrigation fluid is used for flushing away any blood clots that may develop in the blood vessel during the surgery. Further, continuous irrigation and suction inside the blood vessel prevents formation of any new clots inside the vessel.

Outer lumens 206 and 208 are interconnected at the distal and the proximal end of the multi-lumen tube 102. Outer lumens 206 and 208 are used for carrying out suction at the affected site in the blood vessel.

Though functionally only two outer lumens are necessary, one each for irrigation and suction, three outer lumens are provided. The presence of three outer lumens, i.e. outer lumens 204, 206 and 208, gives multi-lumen tube 102 greater uniform flexibility. By having three radial walls, preferably equally spaced, in the annulus between center lumen 202 and outer wall of multi-lumen tube 102, multi-lumen tube 102 does not exhibit greater stiffness in any one plane.

In one embodiment of the present invention, multi-lumen tube 102 has a circular diameter. This diameter is equal to 0.156 inch and the length of multi-lumen tube 102 is 48 inches. The material of multi-lumen tube 102 is Perbax 7033 (Polyether block amide). The diameter of center lumen 202 is equal to 0.042 inch. Center lumen 202 extends out of multi-lumen tube 102 by 0.170 inch on both ends. It may be noted by one skilled in the art that the above values are exemplary and do not limit the invention to these values.

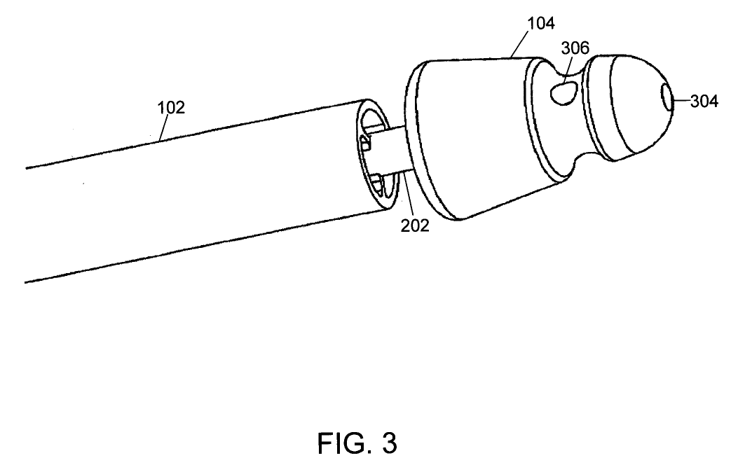

FIG. 3 is an isometric view of the preferred tip device 104 coupled to multi-lumen tube 102, in accordance with the present invention. Tip device 102 has a proximal and a distal end. The proximal end of tip device 104 is attached to the protruding section of center lumen 202 on the distal end of multi-lumen tube 100. As shown in FIG. 3, the diameter of tip device 104 at the proximal end is greater than its diameter at the distal end. This gives tip device 104 a tapering (conical) shape, and hence enables catheter 100 to move in the blood vessel with greater ease. Tip device 104 also comprises a coaxial port 304 on the distal end. Port 304 allows the guide wire to pass through tip device 104 and enter center lumen 204, during the insertion of catheter 100 into the blood vessel. Further, tip device 104 has a circumferential depression between the proximal and distal end. Tip device 104 comprises one or more radial ports in the circumferential depression. Though only one such radial port, port 306, is shown in FIG. 3, it must be apparent to one skilled in the art that there can be several such radial ports. Port 306 provides an outlet in tip device 104 for the sclerosing agent.

The design of tip device 104 helps in providing uniform and complete sclerosis of the blood vessel. This is achieved by the help of a circumferential depression present on tip device 104. When tip device 104 is inserted in the blood vessel, the blood vessel's diameter is completely filled with tip device 104. In such situation, the presence of the circumferential depression allows a cavity to be formed between the inner surface of the blood vessel and tip device 104. This cavity is filled with the sclerosing agent flowing out of port 306. This pool of sclerosing agent hence generated is in contact with the complete circumference of the blood vessel, and hence provides uniform and complete sclerosis of the blood vessel.

In one embodiment of the present invention, tip device 104 is made of stainless steel. The proximal end of tip device 104 is attached to center lumen 202 using a #1-64 UNC screw thread.

In another embodiment of the invention, tip device 104 is made of a plastic material. In this embodiment the proximal end of tip device 104 is attached to center lumen 202 using an adhesive. This adhesive may be cyanoacrylate, UV-cured epoxy and the like.

FIG. 3 also shows the openings in the side wall of the multi-lumen tube 102 at the distal end. One of these openings in the side wall corresponding to outer lumen 204 allows the irrigation fluid to flow out of outer lumen 204 into the blood vessel. The other two side wall openings correspond to lumens 206 and 208 and allow suction of fluids from the blood vessel.

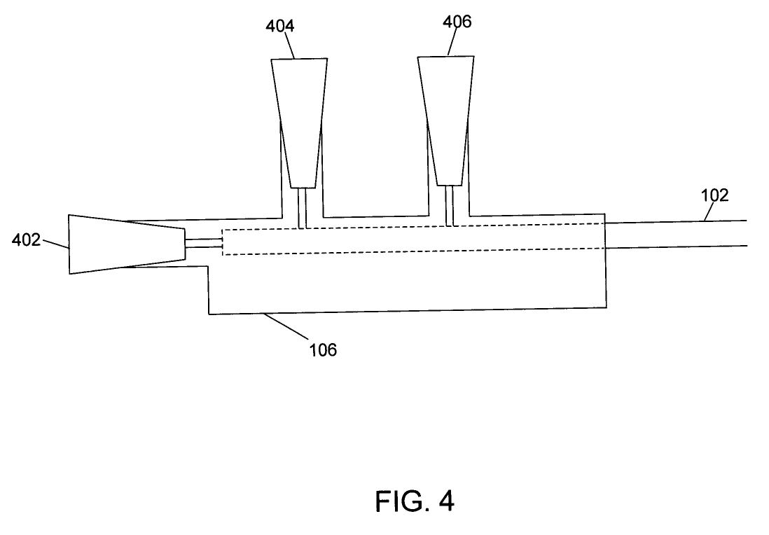

FIG. 4 is a representation of fluid porting device 106 coupled to multi-lumen tube 102, in accordance with one embodiment of the present invention. Fluid porting device 106 is attached to the proximal end of multi-lumen tube 102. Fluid porting device 106 provides ports for accessing each of the fluid passages in multi-lumen tube 102, i.e. center lumen 202 and outer lumens 204, 206 and 208. The ports in fluid porting device 106 are attached to center lumen 202 and outer lumens 204, 206 and 208 by cutting circular scallops through the outer wall of the catheter 100. As shown in FIG. 4, fluid porting device 106 comprises 3 ports; a sclerosing port 402, an irrigation port 404 and a suction port 406. Sclerosing port 402 is connected to center lumen 202 such that sclerosing port 402 is aligned with the tubing axis on the proximal end of multi-lumen tube 102. Irrigation port 404 is connected to outer lumen 204. Suction port 406 is connected to outer lumens 206 and 208. Irrigation ports 404 and suction port 406 are normal to the axis of multi-lumen tube 102.

Having disclosed the catheter system, a method for operating catheter system 100 is disclosed hereinafter. As an example of performing sclerotherapy on an affected blood vessel, a method for performing sclerosis of the long saphenous vein (LSV) has been described. It should be apparent to one skilled in the art that similar to the LSV, the sclerotherapy may be performed on any blood vessel.

FIG. 5 is a flowchart illustrating the method of performing sclerotherapy on the LSV using the catheter system, in accordance with one embodiment of the present invention. The sclerotherapy comprises four main steps as described in the flowchart. At step 502, catheter 100 is inserted into the LSV. Thereafter, at step 504, appropriate pressure is applied on the LSV. The pressure is applied on the LSV in order to remove blood from the LSV and to prevent the flow of blood in the treated section of the LSV. Also, application of pressure prevents the sclerosing agent from entering into the perforator veins. Subsequently, at step 506, irrigation and suction are maintained in the LSV. Also, the sclerosing agent is infused into the LSV along with gradual withdrawal of catheter 100 from the LSV. Lastly, at step 508, catheter 100 is completely withdrawn from the LSV. Each of the above mentioned steps are discussed in further details hereinafter.

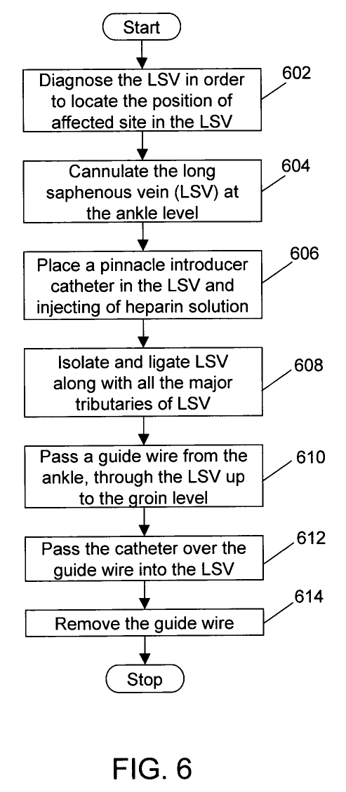

FIG. 6 is a flowchart illustrating the method of inserting the catheter system in the blood vessel, in accordance with one embodiment of the invention. Before the LSV is operated upon, at step 602, the affected site in the LSV is diagnosed in order to locate the exact position of the affected site in the LSV. This diagnosis may be done by using methods like simple clinical tests, doppler ultra sonography, venography and other relevant techniques known in the art. At step 604, the LSV is cannulated at the ankle level. Thereafter, at step 606, a pinnacle introducer catheter is placed in the LSV and heparin solution is injected through the pinnacle introducer catheter. The pinnacle introducer catheter is used in order to facilitate access to the LSV. The heparin solution is introduced in order to prevent formation of any thrombus inside the LSV. In one embodiment of the present invention, a five French pinnacle introducer catheter is used. At step 608, the LSV is ligated and isolated along with all the major tributaries of the LSV by making an incision at the groin level. Subsequently, at step 610 a guide wire is introduced in the LSV at the ankle level. The guide wire is passed through the LSV to the groin level. After the guide wire is passed through the LSV, the proximal end of catheter 100 is introduced into the LSV at the groin level, at step 612. Catheter 100 is introduced such that the guide wire enters catheter 100 through axial port 304. As catheter 100 is passed through the LSV, the guide wire passes through center lumen 204. The guide wire facilitates easy sliding of catheter 100 into the LSV. According to the position of the affected area in the LSV, catheter 100 is inserted to a pre-measured length. Catheter 100 may also be felt by hand in order to determine the position of tip device 104 in the LSV. The position of tip device 104 in the LSV may also be determined by shining a strong beam of light on the leg, particularly on the portion where the LSV exists. Thereafter, at step 614, the guide wire is removed from the LSV.

External pressure is applied and maintained on the LSV throughout the surgery. This is done in order to prevent the sclerosing agent from flowing into the perforator veins connected to the LSV. The pressure is applied through a pressure applying device. The preferred medium of applying pressure is a saphenous vein compressor, disclosed earlier in U.S. patent application Ser. No. 10/400,901, filed on Mar. 27, 2003, titled “Vein Compressor Device” that is incorporated herein by reference. Other devices for applying pressure on the LSV may include an elastic rubber esmark bandage, towel wraps, gauze bandage wraps, support stockings or hand pressure. Typically a pressure of around 70 to 80 mm of Hg is applied on the LSV during sclerotherapy.

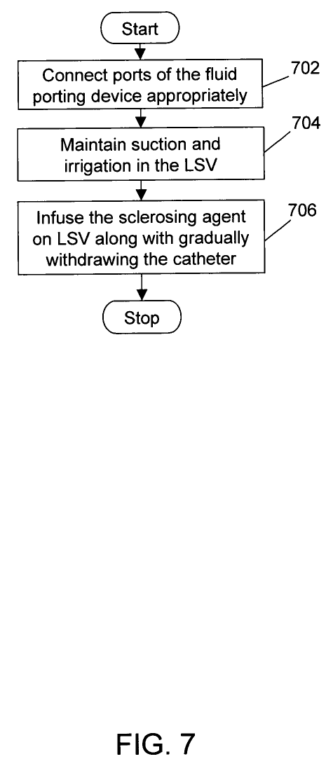

FIG. 7 is a flowchart illustrating the method of operating the catheter system while the sclerosis is being performed in the LSV. Once tip device 104 reaches at the affected position, step 702 is performed. At step 702, sclerosing port 402, irrigation port 404 and suction port 406 are connected appropriately. For example, sclerosing port 402 and irrigation port 404 are attached to separate fluid injecting devices, and suction port 406 is attached to wall suction.

Thereafter, at step 704, irrigation and suction are maintained at affected site in the LSV. Subsequently, at step 706, the sclerosing agent is gradually infused in the LSV and catheter 100 is simultaneously withdrawn gradually from the LSV.

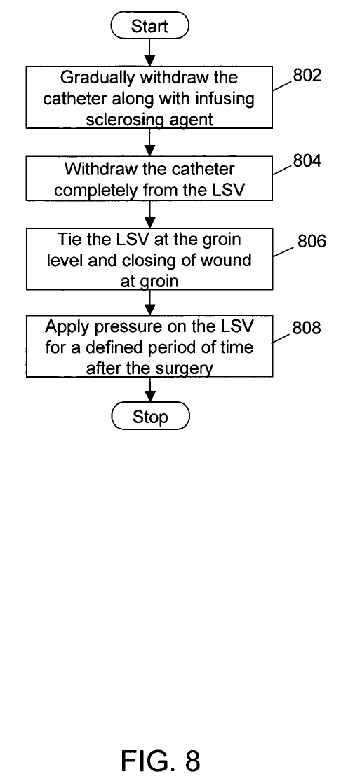

FIG. 8 is a flowchart illustrating the method of withdrawing the catheter system from the blood vessel. At step 802, withdrawal of catheter 100 is continued slowly with infusion of sclerosing agent. Thereafter, catheter 100 is completely withdrawn from the LSV, at step 804. Once catheter 100 is completely withdrawn, the LSV is tied at the groin level and the wound is closed, at step 806.

After the sclerotherapy is performed, pressure is maintained on the LSV. The patient is sent back with the pressure applying device on his/her leg. Typically a pressure of 70 to 80 mm of Hg is applied for 10 minutes after the surgery. Thereafter, the pressure is reduced to 35 to 40 mm of Hg and continued for next 48 to 72 hours. Application of pressure after the surgery prevents blood to flow in the treated section of the LSV and gives time to achieve complete sclerosis of the LSV.

Though only specific steps of carrying out the surgery for sclerosis have been described, it should be obvious to one skilled in the art that other essential steps of the surgery are also performed along with those mentioned herein. Such steps may include anesthetizing the patient before the surgery.

The system and method as described above has a number of advantages. The catheter system has uniform flexibility in all planes. This makes the catheter system easy to use. Moreover the catheter system is simple in design that makes the system convenient to use. The simplicity of the design also makes the catheter cost effective.

The catheter system has a unique tip design that enables the catheter to perform sclerosis uniformly and completely on the inner wall of the affected blood vessel.

While the preferred embodiments of the invention have been illustrated and described, it will be clear that the invention is not limited to these embodiments only. Numerous modifications, changes, variations, substitutions and equivalents will be apparent to those skilled in the art without departing from the spirit and scope of the invention as described in the claims.

What is claimed is:

1. A catheter system suitable for sclerosing a blood vessel, the catheter system comprising:
a. a multi-lumen tube for carrying a sclerosing agent, the multi-lumen tube having a proximal end and a distal end, the multi-lumen tube comprising:
i. a center lumen, the center lumen being used for carrying the sclerosing agent through the multi-lumen tube, wherein the center lumen is coaxial to the multi-lumen tube; and
ii. at least two outer lumens which are isolated from the center lumen and disposed in the annulus between the center lumen and outer wall of the multi-lumen tube, the outer lumens being used for transporting fluids; and
b. a tip device coupled to the distal end of the multi-lumen tube, the tip device being used for infusing the sclerosing agent into the blood vessel.

2. The catheter system according to claim 1 further comprising a fluid porting device, the fluid porting device being attached to the proximal end of the multi-lumen tube, the fluid porting device being used for introducing and withdrawing fluids through the multi-lumen tube.

3. The catheter system according to claim 1 wherein the outer lumens comprise three lumens which are equally spaced in the annulus between the center lumen and the outer wall of the multi-lumen tube.

4. The catheter system according to claim 3 wherein two outer lumens out of the three outer lumens are inter-connected at the proximal end and the distal end of the multi-lumen tube.

5. The catheter system according to claim 1 wherein the tip device is conical in shape.

6. The catheter system according to claim 1 wherein the tip device comprises a coaxial port to allow passing of a guide wire through the tip device.

7. The catheter system according to claim 1 wherein the tip device comprises a circumferential depression, the circumferential depression having at least one radial port for infusing the sclerosing agent.

8. A catheter system suitable for sclerosing a blood vessel, the catheter system comprising a multi-lumen tube, the multi-lumen tube having a proximal end and a distal end, the multi-lumen tube comprising:
i. a center lumen, the center lumen being used for carrying a sclerosing agent; and
ii. at least two lumens in the annulus between the center lumen and the outer wall of the multi-lumen tube which are isolated from the center lumen, wherein the lumens are used for transporting irrigation fluid.

9. The catheter system according to claim 8 wherein the center lumen is coaxial with the multi-lumen tube.

10. The catheter system according to claim 8 wherein the outer lumens comprise three equally spaced lumens disposed in the annulus between the center lumen and the outer wall of the multi-lumen tube.

11. The catheter system according to claim 10 wherein two outer lumens out of the three outer lumens are inter-connected at the proximal end and the distal end of the multi-lumen tube.

12. A sclerosing system suitable for sclerosing a blood vessel, the sclerosing system comprising a catheter, the catheter comprising:
a. a multi-lumen tube, the multi-lumen tube having a proximal end and a distal end, the multi-lumen tube comprising:
i. a center lumen, the center lumen being coaxial to the multi-lumen tube wherein the center lumen is used for carrying a sclerosing agent; and
ii. three equally-spaced lumens in the annulus between the center lumen and the outer wall of the multi-lumen tube, wherein the three equally-spaced lumens are used for transporting fluids;
b. a tip device coupled to the center lumen on the distal end of the multi-lumen tube, the tip device being used for infusing the sclerosing agent into the blood vessel; and
c. a fluid porting device coupled to the proximal end of the multi-lumen tube, the fluid porting device being used for porting the sclerosing agent and the irrigation fluid into the multi-lumen tube, the fluid porting device also providing suction of the fluids through the multi-lumen tube.

13. The sclerosing system according to claim 12 wherein the sclerosing system further comprising a saphenous vein compressor, the saphenous vein compressor being used for applying pressure on the affected blood vessel.

14. The sclerosing system according to claim 12 wherein the tip device is conical in shape.

15. The sclerosing system according to claim 12 wherein the tip device comprises a coaxial port to allow passing of a guide wire through the tip device.

16. The sclerosing system according to claim 12 wherein the tip device comprises a circumferential depression, the circumferential depression having at least one radial port for infusing the sclerosing agent.

17. A method for sclerosing a blood vessel using a catheter system, the catheter system comprising a multi-lumen tube, a tip device coupled to the distal end of the multi-lumen tube, and a fluid porting device coupled to the proximal end of the multi-lumen tube, the multi-lumen tube comprising a center lumen and at least two outer lumens in the annulus between the center lumen and the outer wall of the multi-lumen tube, the method comprising the steps of:
a. inserting the catheter system into the blood vessel, the catheter system being inserted till the tip device reaches the affected site.
b. maintaining appropriate pressure on the blood vessel using a pressure applying device;
c. infusing a sclerosing agent through the tip at the affected site; and
d. removing the catheter system from the blood vessel.

18. The method as recited in claim 17 wherein the step of inserting the catheter comprises the steps of:
a. inserting a guide wire into the blood vessel; and
b. passing the catheter system into the blood vessel following the guide wire, such that the guide wire passes through the multi-lumen tube.

19. The method according to claim 17 further comprising the step of carrying the sclerosing agent through the multi-lumen tube.

20. The method according to claim 19 wherein the step of carrying the sclerosing agent is performed such that the sclerosing agent is carried through the center lumen of the multi-lumen tube.

21. The method according to claim 17 further comprising the step of carrying irrigation fluids through the multi-lumen tube.

22. The method according to claim 21 wherein the step of carrying the irrigation fluids is performed such that the irrigation fluids are carried through one of the outer lumens of the multi-lumen tube.

23. The method according to claim 17 further comprising the step of sucking fluids from the blood vessel through the multi-lumen tube.

24. The method according to claim 23 wherein the step of sucking is performed such that fluids are sucked through two of the outer lumens of the multi-lumen tube.

25. The method according to claim 17 wherein the step of maintaining pressure is performed using a saphenous vein compressor.
